(19) United States
(12) Patent Application Publication
(10) Pub. No.: US 2004/0132365 A1
(43) Pub. Date: Jul. 8, 2004

(54) Intermediate composite part for forming reinforcement prosthesis

(75) Inventors: Michel Therin, Lyon (FR); Philippe Gravagna, Irigny (FR)

Correspondence Address: OLIFF & BERRIDGE, PLC, P.O. BOX 19928, ALEXANDRIA, VA, 22320, US

(73) Assignee: SOFRADIM PRODUCTION, Trevoux (FR)

(21) Appl. No.: 10/690,532

(22) Filed: Oct. 23, 2003

Related U.S. Application Data
(60) Provisional application No. 60/423,378, filed on Nov. 4, 2002.

(51) Int. Cl. B32B 27/12; A61F 2/02; B32B 27/04; B32B 5/02

(52) U.S. Cl. 442/43; 623/23.74; 623/23.75; 428/297.4; 442/304

(57) ABSTRACT

The present invention relates to an intermediate composite part (1) designed for forming a composite reinforcement prosthesis and comprising:

i) a porous textile support (2) which includes an arrangement of threads each composed of at least one filament of nonabsorbable polymer material, said textile support defining the free edge or edges (7) of the part, and

ii) a hydrophilic absorbable material (3) covering said textile support, at least on one side of the latter, extending across the surface of what is called a protected zone (4) and creating, on each side of the latter, two unprotected zones (5, 6) of the textile support which are limited by said free edges and are devoid of any of said absorbable material.

It also relates to a process for preparing such a part.

This nonprovisional application claims the benefit of U.S. Provisional Application No. 60/423,378, filed Nov. 4, 2002.

### FIELD OF THE INVENTION

The present invention relates to an intermediate composite part for forming reinforcement prostheses for tissue structures.

### DESCRIPTION OF THE PRIOR ART

Reinforcement prostheses generally serve to support the tissues and can be used in different areas of the body. These reinforcement prostheses may be designed for temporary implantation. In these cases, they are made of an absorbable material. They may, however, be designed for permanent implantation and they then consist, for example, of a textile support made of a nonabsorbable material.

Textile supports are intrinsically adhesiogenic and fibrotic, irrespective of the nature of the tissues with which they are placed in contact. This property, when considered with respect to support tissues (muscles, aponeuroses, fascias, etc.), in fact constitutes an indispensable prerequisite for the quality of the result. By contrast, with respect to other more fragile structures, the presence of a textile support at the initial phase of cicatricial inflammation promotes the creation of dense fibrous connections where previously there were only loose connections, such as those provided by the interstitial connective tissues for the extraperitoneal organs, and where there was no connection, for the intraperitoneal organs. For this reason, the porous nature of textile supports is often the cause of the development of postsurgical adhesions and erosions.

One way of overcoming this problem is for the face of the prosthesis on which the formation of these adhesions or these erosions is not wanted to be made smooth, preferably by covering said face with an absorbable material.

For example, in parietal surgery, reinforcement prostheses are implanted with the aim of repairing the walls of the abdominal cavity which have been damaged by hernias or eventrations. If implanted in the abdominal cavity in an intraperitoneal location, the reinforcement prosthesis must therefore preferably have a surface, toward the wall, that encourages cell colonization for effective surgical reconstruction and, on the side toward the viscera, a smooth face which does not favor formation of postsurgical adhesions. In such a case, the reinforcement prostheses are preferably in the form of a porous textile of which one face is covered superficially by an absorbable layer or membrane in order to avoid the formation of postsurgical adhesions, the other face being left free for intimate and early tissue integration of the wall that is to be supported. WO99/06080 describes such prostheses.

Another example is the case of surgical treatment for supporting tissues or for repairing prolapses. The tissues involved in these treatments, called extraperitoneal tissues, are particularly exposed since they can come into contact with both faces of the prosthesis. These tissues are, for example, hollow viscera such as the bladder, vagina, uterus or rectum, or natural ducts of the organism, such as the urethra. The esophagus, trachea, blood vessels, nerves, tendons, and dura mater constitute other examples of fragile structures which it is necessary to protect when using porous reinforcements in their proximity. For fragile tissues such as these, the fibrous connections subsequent to the initial cicatricial inflammation may be aggravated by erosion or fistulas. In this case, it is necessary, during the initial phase of cicatrization, to protect these surrounding fragile tissues by covering both surfaces of the prosthesis with a smooth material.

Surgical treatments to support tissues or repair prolapses involve supporting these fragile tissues (bladder, vagina, uterus, rectum) while connecting them to stronger tissues situated in anatomically stable zones. Thus, the part of the prosthesis in contact with the fragile tissues must have smooth faces, while the parts of the prosthesis in contact with the strong, more distant tissues are preferably porous in order to permit better anchoring of the prosthesis. In this regard, WO00/02031 describes a prosthesis designed for treatment of incontinence in females, the central part of which prosthesis is covered with nonabsorbable hydrophobic silicone, and the lateral anchoring parts of which are knitted or nonwoven lattices. However, such a prosthesis is not especially desirable for protecting fragile structures, because of the hydrophobic and nonabsorbable nature of the silicone.

Moreover, for hygiene reasons or for making the surgeon's task easier, it may be desirable to cover only part of a prosthesis. For example, it may be desired to temporarily stiffen part of a prosthesis by covering it with a continuous film for use in laparoscopic surgery.

Finally, the reinforcement prostheses must also be adapted to the size and internal morphology of the patient.

Thus, it is clear that there are as many shapes and types of prosthesis as there are pathologies and patients. However, it would be tedious and indeed dangerous to completely produce a prosthesis in situ during the operation. This would take up an enormous amount of time, would prolong the period of anesthesia necessary for the operation, and would increase the risks of complications.

Therefore, it would be advantageous if the surgeon operating on a hernia or a prolapse were provided with an intermediate composite part from which he could easily and quickly cut out a reinforcement prosthesis adapted to the morphology and to the pathology of the patient on whom he is operating.

### SUMMARY OF THE INVENTION

The present invention therefore relates to an intermediate composite part designed for forming a composite reinforcement prosthesis and comprising:

i) a porous textile support which includes an arrangement of threads each composed of at least one filament of nonabsorbable polymer material, said textile support defining the free edge or edges of the part, and

ii) a hydrophilic absorbable material covering said textile support, at least on one side of the latter, extending across the surface of what is called a protected zone and creating, on each side of the latter, two unprotected zones of the textile support which are limited by said free edges and are devoid of any of said absorbable material.

The present invention also relates to the use of an intermediate composite part, such as that above, for obtaining a composite reinforcement prosthesis.

The present invention relates further to a process for obtaining a composite reinforcement prosthesis, wherein:

i) an intermediate composite part as described above is obtained,

ii) the composite part is cut out along a cutting line defining in this part at least two zones of tissue attachment, on each side of a zone of tissue integration, the attachment zones being cut out from the two unprotected zones, respectively, and the integration zone being cut out from the protected zone.

By virtue of the invention, the surgeon is able, while operating, to cut out the prosthesis best adapted to the morphology of the patient, taking into account the pathology for which he is in the process of operating on the patient.

The term “porous textile” as used in the present invention is to be understood as a textile support having empty spaces in the form of interstices and/or volumes. More precisely, the porous textile support is composed of an arrangement of threads defining a microporous texture and/or a macroporous texture.

The arrangement of threads in question is an interlacing which may be ordered, for example woven, according to any suitable weave, or knitted, or else unordered, for example nonwoven. In a preferred embodiment of the invention, the arrangement of threads constitutes a knitted structure. Each thread in question comprises at least one continuous or noncontinuous filament made of nonabsorbable polymer material; each thread can comprise other threads or filaments, for example made of absorbable polymer material.

The term “polymer material” is to be understood as any material, alone or in combined form, comprising a synthetic or natural polymer obtained for example by polymerization or copolymerization. The nonabsorbable polymer material according to the invention can be a polypropylene or alternatively a polyester of the polyethylene terephthalate type.

The microporous texture comprises at least the interstices located between at least two threads at the sites of contact of one thread with at least one other thread. In the case where at least one thread comprises several filaments which may or may not be joined together, the microporous texture additionally comprises the interstices between filaments of the same thread.

The microporosity of the textile support can thus be defined as being the sum of i) the interstices, if any, located between at least two filaments within the same thread and ii) the interstices located between at least two threads at the sites of contact of one thread with at least one other thread.

The macroporous texture or macroporosity comprises the volumes whose surface S is defined by the empty spaces between at least two threads, away from their sites of contact, and whose height H is defined by the thickness of the textile support. According to the present invention, the textile support is considered as being flat when it constitutes a two-dimensional structure, preferably when its thickness is less than or equal to 5 times the mean diameter of the threads of which it is made up. This will give a two-dimensional macroporosity or two-dimensional macroporous texture. In the case where the textile support constitutes a three-dimensional structure, preferably when the thickness of the textile support is strictly greater than 5 times the mean diameter of the threads constituting the support, this will give a three-dimensional macroporosity or three-dimensional macroporous texture.

In a preferred embodiment of the invention, the textile support constitutes a two-dimensional structure.

The textile support is made of a nonabsorbable polymer material commonly used in surgery. This nonabsorbable polymer material is preferably chosen from the group comprising polypropylenes, polyesters such as polyethylene terephthalates, polyamides and/or their mixtures. In a preferred embodiment of the invention, this polymer material is polypropylene. Examples of a polypropylene-based textile support suitable for the present invention are the product sold under the brand name Parietene® by Sofradim, the product sold under the brand name Prolene® by Ethicon, or the product sold under the brand name Marlex® by Bard.

The hydrophilic absorbable material according to the invention is chosen from the group formed by the collagens, polysaccharides, and their mixtures. Among the collagens which can be used according to the invention, the following may be mentioned:

1) collagen whose helix structure is at least partially denatured by heat, without hydrolytic breakdown, and whose method of preparation is described in WO99/06080,

2) native collagen, unheated, formed into a film, with or without glycerin, crosslinked by gamma irradiation or by other means chemical or physical,

3) and/or their mixtures.

Among the polysaccharides which can be used as absorbable hydrophilic material according to the invention, mention may be made of oxidized cellulose, hyaluronic acid, starch, chitosan, crosslinked dextrans and/or their mixtures. All these materials are well known to the person skilled in the art. An example of an oxidized cellulose suitable for the present invention is the product sold under the brand name Interceed® by Ethicon. An example of hyaluronic acid suitable for the present invention is the product sold under the brand name Hyalobarrier® by Fidia Advanced Biopolymers, or the product sold under the brand name Seprafilm® by Genzyme.

The hydrophilic absorbable material covers the textile support on one of its sides or on both of its sides. According to the invention, “protected zone” is defined as the zone of the textile support covered by the hydrophilic absorbable material.

The absorbable material can cover the textile support by means of a membrane linked to the textile support by bonding and/or partial impregnation or alternatively by means of a coating which occludes the microporosity. The coating may, if appropriate, additionally occlude the macroporosity of the textile support on a zone referred to as occluded zone.

In the case where the absorbable material covers the textile support by means of a membrane, the latter is preferably linked at least superficially to the textile support, more preferably over a certain thickness by capillary absorption of the threads constituting the textile support. The membrane of absorbable material is preferably continuous, smooth and nonporous. It can form a plane layer with a thickness of up to several millimeters.

The composite part according to the invention can be obtained by applying to the side of the textile support to be covered a layer of a solution of absorbable material which will form the membrane after gel formation and drying. Another way of proceeding is to prepare a first layer of a solution of absorbable material in a dish. After this first layer has cooled and formed a gel, a second layer from the same solution is applied to its surface. That part of the side of the textile support which is to be covered is then applied to the second layer, before gel formation, in such a way that the anchoring takes place during the drying of this second layer. A plane membrane is thus obtained having a thickness which can be up to several millimeters. If both sides of the textile support are to be covered, the same operation is repeated on the other side of the support.

The intermediate composite part can also be prepared by spraying the solution of hydrophilic absorbable material onto all or part of the textile support, preferably with the aid of a stencil or a protective mask. The spraying can be carried out on a first face of the textile. After 15 minutes to 2 hours of maturation of the gel formed on the surface of the textile, the solution can be sprayed onto the other face. After the second spraying operation, the whole arrangement is left to dry under a flow of sterile air. After maturation, the whole arrangement is sterilized using ethylene oxide.

In the case where the covering is a coating, the hydrophilic absorbable material coats the protected zone of the textile support by forming a film enveloping and penetrating into the arrangement of threads, which occludes the microporous structure, in other words at least the microporosity of the support, but without forming a thick layer or membrane covering at least one face of the textile support. The film directly or indirectly coats at least each thread completely, forming a coating. Moreover, in the case where each thread of the support is formed by a single filament, the absorbable film fills and thus occludes all the interstices located between at least two threads at the sites of contact of one thread with at least one other thread. In the case where at least one thread comprises several filaments, which may or may not be joined to one another, the film fills and thus likewise occludes all the interstices between filaments of the same thread.

In a preferred embodiment of the invention, the film which coats the textile support as described in the above paragraph is noncontinuous and preserves the macroporous texture of the textile support. The film then coats each thread and leaves free the volumes defining the macroporosity. Thus, the reinforcement preserves a pronounced macroporosity for rapid mechanical anchoring and immediate cell recolonization. Such coating with a noncontinuous film also makes it possible to temporarily stiffen the reinforcement and facilitate its handling by the surgeon. This latter property is particularly advantageous in laparoscopic surgery.

The film of absorbable material preferably has a thickness of less than or equal to 500 microns, and still more preferably in the range of 10 to 100 microns.

In another embodiment of the invention, the film can also occlude the macroporosity of the textile support over part of the protected zone. According to the invention, “occluded zone” is defined as the part of the protected zone for which the macroporosity is occluded. In this occluded zone, the film fills and occludes the two-dimensional or three-dimensional volumes defining the macroporosity. It is therefore continuous.

When the cover is a coating, the intermediate composite part according to the invention can be prepared according to the process comprising the following steps:

i) preparing a solution of a hydrophilic absorbable material, in the fluid or liquid state,

ii) immersing that part of the textile support which is to be covered in this solution,

iii) removing the part of the textile support impregnated with solution and drying it.

The solution of hydrophilic absorbable material must be sufficiently fluid so that it impregnates the textile support in order to occlude the microporosity. This solution can be heated to a temperature below 50° C. In order to facilitate this impregnation step, solution A preferably has a viscosity of less than or equal to 1000 centipoises, and still more preferably from 50 to 200 centipoises, this viscosity being measured with the aid of a CONTRAVES-TV viscosimeter, for example, at the chosen temperature of less than 50° C. Those parts of the textile support which are not intended to be coated can be covered with a protective membrane during the immersion step. In the case where the coated zone represents a central band of the textile support, the support can also be folded beforehand into a U shape, the part to be coated being the horizontal bar of the U, and the part to be coated can thus be immersed in the solution of absorbable material while preserving the lateral parts (vertical bars of the U).

In a preferred embodiment of the invention, the intermediate composite part is in the form of a rectangular part and the protected or occluded zone represents a central band of this part.

By virtue of the invention, the practitioner can freely form reinforcement prostheses by cutting out bands of suitable geometry from the intermediate composite part and placing them in such a way as to obtain a support which is nonaggressive, by virtue of the protected or occluded zone, with respect to the fragile tissue structures, and a rapid anchoring in the unprotected zones away from the fragile structures.

Such prostheses have zones for immediate attachment to permit effective suspension immediately upon implantation, by retraction of the interstitial connective tissues, fascias and aponeuroses at the free edges of the threads, relatively fibrotic zones for stable mechanical anchoring at the unprotected part of the textile support, and zones of nonaggressive and minimally fibrotic integration in the central protected or occluded zone of the reinforcement.

A reinforcement prosthesis can be formed from the intermediate composite part according to the invention by means of the following process:

i) an intermediate composite part as described above is obtained,

ii) the composite part is cut out along a cutting line defining in this part at least two zones of tissue attachment, on each side of a zone of tissue support, the attachment zones being cut out from the two unprotected zones, respectively, and the tissue support zone being cut out from the protected zone.

In a preferred embodiment of the invention, the cutting line defines a strip with straight parallel edges. In another embodiment of the invention, the cutting line defines a strip with parallel edges curved in an arch. In another embodiment of the invention, the cutting line defines a strip with nonparallel edges, which is bulged in the central region, in order to support the prolapsed structures on a large surface, and is narrower at the ends in order to constitute anchoring suspension strips. These strips are of adaptable width and length.

### BRIEF DESCRIPTION OF THE DRAWING

The present invention will be better understood by referring to the following figures:

FIG. 1 is a plan view showing an intermediate composite part according to the invention, of rectangular shape and with its protected zone in the shape of a central band,

FIG. 2 is a plan view showing an intermediate composite part according to the invention and the shapes of prostheses which can be cut out.

### DESCRIPTION OF THE PREFERRED EMBODIMENTS

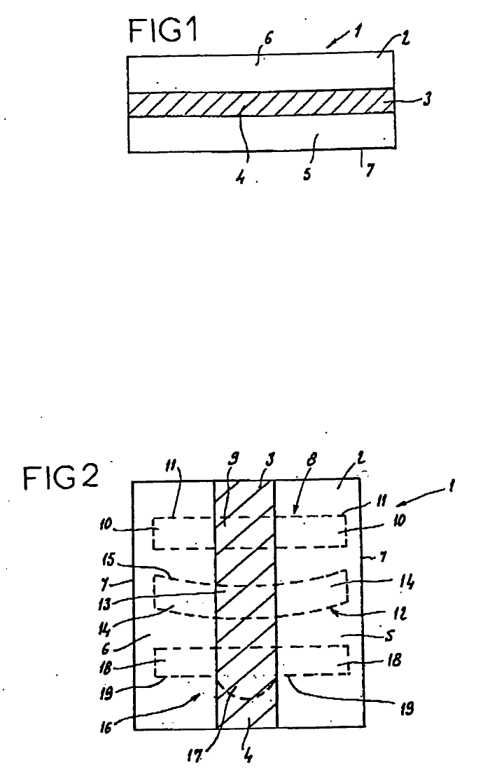

Referring to FIG. 1, an intermediate composite part 1 of rectangular shape is shown, comprising a textile support 2 whose central part 4, here in the shape of a central band, is protected by an absorbable material 3. This absorbable material can cover the part 4 as a film or as a membrane. The parts 5 and 6 of the textile support 2 limited by their free edges 7 are devoid of absorbable material.

FIG. 2 shows a part, according to FIG. 1, indicating the cuts along which prostheses can be cut out. Thus, it is possible to cut out a strip 8 with parallel edges which has a protected central zone 9 and two lateral parts 10 devoid of absorbable material. Once cut out, the strip 8 constitutes a composite reinforcement prosthesis whose lateral parts 10 define relatively fibrotic tissue attachment zones for stable mechanical anchoring, while the central part 9 defines a zone of minimally fibrotic and nonaggressive tissue support. The free edges 11 define a zone of immediate attachment to permit effective suspension from the moment of implantation. It is also possible to cut from the part 1 a strip 12 with parallel edges curved in an arch, which strip 12 has a central protected part 13 and two lateral parts 14 devoid of absorbable material. Once cut out, the strip 12 constitutes a composite reinforcement prosthesis whose lateral parts 14 define relatively fibrotic tissue attachment zones, while the central part 13 defines a zone of minimally fibrotic and nonaggressive tissue support. The free edges 15 define a zone of immediate attachment. It is also possible to cut from the part 1 a strip 16 with nonparallel edges which has a protected, bulged central part 17 and two lateral parts 18 which are narrower and are devoid of absorbable material. Once cut out, the strip 16 constitutes a composite reinforcement prosthesis whose narrower lateral parts 18 define relatively fibrotic tissue attachment zones, while the bulged central part 17 defines a zone of minimally fibrotic and nonaggressive tissue support. The free edges 19 define a zone of immediate attachment.

### EXAMPLE 1

A textile support of rectangular shape and measuring 25×20 cm² is produced.

A solution is prepared comprising collagen modified by oxidative cutting and heating, polyethylene glycol and glycerin. The weight concentrations of collagen, polyethylene glycol and glycerin are preferably between about 2 and 9% for the collagen, between about 0.6 and 3% for the polyethylene glycol, and between about 0.3 and 1.2% for the glycerin.

This solution has a viscosity of from 50 to 200 centipoises. A central band (6 cm wide) of the textile support is immersed in this solution, then it is withdrawn and left to gel for 30 minutes. This operation is repeated as many times as is necessary to obtain a film with a density of 0.133 g/cm².

An intermediate composite part is obtained whose central band of width 6 cm is an occluded zone according to the invention, and whose lateral parts are devoid of absorbable material.

A strip with parallel edges and measuring 20×4 cm² is cut out from this part in the width direction, giving a composite reinforcement prosthesis whose central part measuring 6×4 cm² is an occluded part, and whose lateral parts, each measuring 7×4 cm², are relatively fibrotic unprotected areas.

### EXAMPLE 2

Starting from the same textile support and the same solution as were described in Example 1, the solution is sprayed onto the central band of the textile support which is to be protected. The spraying is carried out on one face of the textile. After 15 minutes to 2 hours of maturation of the gel formed on the surface of the textile, the solution is sprayed onto the other face. After the second spraying operation, the whole arrangement is left to dry under a flow of sterile air. The quantity of solution necessary to obtain a film with a final density of 0.133 g/cm² is sprayed on. After maturation, the whole arrangement is sterilized with ethylene oxide.

An intermediate composite part is obtained whose central band of width 6 cm is an occluded zone according to the invention and whose lateral parts are devoid of absorbable material.

A strip with parallel edges and measuring 20×4 cm² is cut out from this part in the width direction, giving a composite reinforcement prosthesis whose central part measuring 6×4 cm² is an occluded part, and whose lateral parts, each measuring 7×4 cm², are relatively fibrotic unprotected areas.

1. An intermediate composite part designed for forming a composite reinforcement prosthesis and comprising:
i) a porous textile support which includes an arrangement of threads each composed of at least one filament of nonabsorbable polymer material, said textile support defining the free edge or edges of the part, and
ii) a hydrophilic absorbable material covering said textile support, at least on one side of the latter, extending across the surface of what is called a protected zone and creating, on each side of the latter, two unprotected zones of the textile support which are limited by said free edges and are devoid of any of said absorbable material.

2. The part as claimed in claim 1, wherein the arrangement of threads constitutes a knitted structure.

3. The part as claimed in claim 1, wherein the textile support constitutes a two-dimensional structure.

4. The part as claimed in claim 1, wherein the nonabsorbable polymer material is chosen from the group comprising polypropylenes, polyesters, polyamides and/or their mixtures.

5. The part as claimed in claim 1, wherein the absorbable material is chosen from the group formed by collagens, polysaccharides, and their mixtures.

6. The part as claimed in claim 1, wherein the absorbable material covers the textile support on both of its sides.

7. The part as claimed in claim 1, wherein the absorbable material covers the textile support by means of a membrane linked to the textile support by bonding and/or partial impregnation.

8. The part as claimed in claim 1, wherein the textile support defines a microporosity and a macroporosity.

9. The part as claimed in the claim 8, wherein the absorbable material covers the textile support by means of a coating which occludes the microporosity of the textile support.

10. The part as claimed in claim 9, wherein the coating also occludes the macroporosity of the textile support in a zone called the occluded zone.

11. The part as claimed in claim 1, wherein said part is rectangular, and wherein the protected zone represents a central band of said part.

12. The part as claimed in claim 10, wherein said part is rectangular, and wherein the occluded zone represents a central band of said part.

13. Use of the intermediate composite part as claimed in claim 1, for obtaining a composite reinforcement prosthesis.

14. Process for obtaining a composite reinforcement prosthesis, wherein:
i) an intermediate composite part as claimed in claim 1 is obtained,
ii) the composite part is cut out along a cutting line defining in this part at least two zones of tissue attachment, on each side of a zone of tissue support, the attachment zones being cut out from the two unprotected zones, respectively, and the tissue support zone being cut out from the protected zone.

15. The process as claimed in claim 14, wherein the cutting line defines a strip with straight parallel edges.

16. The process as claimed in claim 14, wherein the cutting line defines a strip with parallel edges curved in an arch.

17. The process as claimed in claim 14, wherein the cutting line defines a strip with nonparallel edges, which is bulged in the central region and narrower at the ends.
